(19) United States
(12) Patent Application Publication
(10) Pub. No.: US 2016/0184331 A1
(43) Pub. Date: Jun. 30, 2016

(54) BASE AND EXTERNAL PREPARATION FOR SKIN

(71) Applicant: KANAE TECHNOS CO., LTD., Kanonji-shi, Kagawa (JP)

(72) Inventors: Yuko KANEMITSU, Takamatsu-shi (JP); Daisuke YASUKI, Kanonji-shi (JP); Yuko FUJITA, Saijyo-shi (JP); Yasushi TSUJIMURA, Kanonji-shi (JP)

(21) Appl. No.: 14/650,701

(22) PCT Filed: Dec. 6, 2013
(86) PCT No.: PCT/JP2013/007185

§ 371 Date: Jun. 9, 2015

(30) Foreign Application Priority Data
Dec. 11, 2012 (JP) 2012-270153

(51) Int. Cl. A61K 31/665; A61K 47/06; A61K 47/24; A61K 47/32; A61K 9/06

(52) U.S. Cl. CPC A61K 31/665; A61K 47/32; A61K 9/06; A61K 47/24; A61K 47/06

(57) ABSTRACT

An object of the present invention is to provide: a novel base that, after application to a skin surface, can rapidly form a coating film and moreover mitigates skin irritation and occurrence of odors; and an external preparation for skin. The base is used in an external preparation for skin that administers an active ingredient transdermally, and is characterized by containing at least an acrylic-based synthetic polymer and a volatile oil, wherein the base is in a liquid or ointment form prior to application to the skin surface, and the volatile oil evaporates after application of the base to form a hydrophobic coating film containing the volatile oil in the range of 0 to 60 wt % with respect to 100 parts by weight of the acrylic-based synthetic polymer.

### TECHNICAL FIELD

The present invention relates to a base used in an external preparation for skin that administers an active ingredient transdermally, and to an external preparation for skin.

### BACKGROUND ART

External preparations for skin, which are dosage forms in which active ingredients are dissolved or dispersed in liquid or ointment (creamy) bases, are applied to skin surfaces to administer active ingredients transdermally.

Transdermal administration of active ingredients takes a long time, and thus the external preparations for skin are required to remain attached to the skin surface for a long time.

An external preparation for skin (bath film agent) that, after application to a skin surface, can form a coating film has been recently developed (see, for example, Patent Document 1).

### PRIOR ART DOCUMENT

### Patent Document

Patent Document 1: Japanese Patent Laid-open Publication No. 2007-015973

### SUMMARY OF THE INVENTION

### Problems to be Solved by the Invention

The external preparation for skin described in Patent Document 1 is a pharmaceutical preparation formed by dissolving nitrocellulose in a solvent, such as 3-methylbutyl acetate, isobutyl acetate, or acetone, and further adding ethyl alcohol thereto. Since nitrocellulose is difficult to handle because of its explosibility, ensuring safety during production of the pharmaceutical preparation is challenging.

Moreover, solvents such as 3-methylbutyl acetate, isobutyl acetate, and acetone are not always preferred ingredients in external preparations for skin because they cause odors and skin irritation.

The present invention has been made in view of the above-described technical problems. An object of the present invention is to provide: a novel base that, after application to a skin surface, can rapidly form a coating film and moreover mitigates occurrence of odors and skin irritation; and an external preparation for skin.

### Solutions to the Problems

In order to solve the above-described technical problems, the base of the present invention (hereinafter referred to as an “inventive base”) is used in an external preparation for skin that administers an active ingredient transdermally, and is characterized by containing at least an acrylic-based synthetic polymer and a volatile oil, wherein the base is in a liquid or ointment form prior to application to a skin surface, and the volatile oil evaporates after application of the base to form a hydrophobic coating film containing the volatile oil in the range of 0 to 60 wt % with respect to 100 parts by weight of the acrylic-based synthetic polymer.

In a preferred aspect of the present invention, the inventive base has hydrophobicity with a weight change rate (X) of 5.0% or less in accordance with the following test.

### <Test>

The weight (D) of the base is measured after immersing a cotton nonwoven fabric (10 cm×10 cm, weight 60 g/m²) in the base and drying the fabric in a 60° C. thermostatic bath for 24 hours. The cotton nonwoven fabric after immersion and drying is next placed in water, stirred for 1 hour, and then taken out of water. After removal of water on the surface of the fabric, the weight (W) of the fabric is measured and the weight change rate (X) is calculated in accordance with the following equation.

Equation: X=(W−D)×100/D.

In a preferred aspect of the present invention, the acrylic-based synthetic polymer in the inventive base is at least one selected from alkyl acrylate copolymer, alkyl acrylate copolymer sodium, alkyl acrylate copolymer ammonium, (alkyl acrylate/diacetone acrylamide) copolymer, and (alkyl acrylate/dimethicone) copolymer.

In a preferred aspect of the present invention, the volatile oil in the inventive base is at least one selected from short chain paraffins and volatile silicone oils.

The external preparation for skin of the present invention (hereinafter referred to as an “inventive external preparation”) is characterized by containing the inventive base and an active ingredient, wherein the base is in a liquid or ointment form prior to application to a skin surface, and the volatile oil evaporates after application of the base to form a hydrophobic coating film containing the volatile oil in the range of 0 to 60 wt % with respect to 100 parts by weight of the acrylic-based synthetic polymer.

In a preferred aspect of the present invention, the inventive external preparation is used in combination with application of foundation on a hydrophobic coating film formed by application of the inventive external preparation to a skin surface.

### Effects of the Invention

The present invention enables rapid formation of a coating film after application to a skin surface as well as mitigation of occurrence of odors and skin irritation.

### EMBODIMENTS OF THE INVENTION

Embodiments of the present invention will be described below by way of examples, but the present invention is not limited to these examples.

### Examples 1 to 7

The compositions of inventive bases according to Examples 1 to 7 (before application) are shown in Table 1.

TABLE 1

| | | Example | | | | | | |
|---|---|---|---|---|---|---|---|---|
| Ingredient (wt %) | | 1 | 2 | 3 | 4 | 5 | 6 | 7 |
| Acrylic-based | Alkyl acrylate copolymer (C10-30) | 40 | | | | | | |
| resin | Alkyl acrylate copolymer Na (C10-30) | | 40 | | | | | |
| | Alkyl acrylate copolymer ammonium (C10-30) | | | 40 | | | | |
| | (Alkyl acrylate/diacetone acrylamide) copolymer (C10-30) | | | | 40 | | | |
| | (Alkyl acrylate/dimethicone) copolymer (C10-30) | | | | | 10 | 40 | 60 |
| Volatile oil | Volatile silicone oil | 60 | 60 | | | | | |
| | (cyclic dimethyl silicone oil) | | | | | | | |
| | Short chain paraffin | | | 60 | 60 | 90 | 60 | 40 |
| | (light isoparaffin (isodecane)) | | | | | | | |

### <Properties of Inventive Bases>

It has been found that the inventive bases according to Examples 1 to 7 all maintain a liquid or ointment form before application to a skin surface, and after application to a skin surface, the volatile oil evaporates to rapidly form a coating film on the skin surface.

The inventive base minimizes occurrence of odors and skin irritation because it contains an acrylic resin as an ingredient for forming a coating film and a volatile oil (short chain paraffin (liquid isoparaffin) or volatile silicone oil) as an ingredient that evaporates upon application to a skin surface.

### <Coating Film>

The inventive bases according to Examples 1 to 7 were applied in a certain amount to the surface of a glass plate and dried in a 35° C. thermostatic bath. The amount of residue of the volatile oil remaining on the coating film formed on the glass surface was measured by comparing the weights of the inventive base at application and after drying. The results showed that, in any of examples, the coating film was formed in which the volatile oil remained in the range of 0 to 60 parts by weight with respect to 100 parts by weight of the acrylic resin. In particular, the coating films formed from the inventive bases according to Examples 3 to 7 containing light isoparaffin as a volatile oil were confirmed to contain the volatile oil in the range of 0 to 30 parts by weight with respect to 100 parts by weight of the acrylic resin.

### <Hydrophobicity of Coating Film>

Next, the coating films formed from the inventive bases according to Examples 1 to 7 were evaluated for their hydrophobicity under the following test conditions.

### <Test>

The weight (D) of the inventive bases according to Examples 1 to 7 is measured after immersing a cotton nonwoven fabric (10 cm×10 cm, weight 60 g/m²) in the bases and drying the fabric in a 60° C. thermostatic bath for 24 hours. The cotton nonwoven fabric after immersion and drying is next placed in water, stirred for 1 hour, and then taken out of water. After removal of water on the surface of the fabric, the weight (W) of the fabric is measured and the weight change rate (X) is calculated in accordance with the equation: X=(W−D)×100/D.

The results of the test showed that the inventive bases according to Examples 1 to 7 all had a weight change rate (X) of 5% or less, and thus exhibited high hydrophobicity. In particular, the inventive bases according to Examples 5 to 7 containing (alkyl acrylate/dimethicone) copolymer as an acrylic resin were confirmed to have very high hydrophobicity with a weight change rate (X) of 2% or less.

For reference, a commercially available hydrophobic base (oleaginous base) was subjected to the same test and found to have a weight change rate (X) of 500% or more.

### Example 8

An inventive external preparation was obtained by incorporating 5 wt % of sodium tocopheryl phosphate (TPNa) as an active ingredient to the inventive base according to Example 6.

The resulting inventive external preparation was evaluated for the skin permeability of the active ingredient in accordance with the following test.

### <Test>

A stratum corneum/epidermis layer of the human skin is cut into a circle with 30 mm in diameter and used for the test. The permeability test is carried out in a non-sealed system using a Franz diffusion cell (effective area 3.14 cm², receptor volume 2.4 mL) as a diffusion cell and a PBS (phosphate buffered saline, pH 7.4) solution as a receptor solution while the diffusion cell is kept at 32° C. The inventive base (1 mL) is applied to the donor side. After 5 minutes, 1 mL of the PBS solution is added to the donor side to wash the layer lightly, and the solution on the donor side is then discarded. This washing procedure is performed another two times (total three times). Thereafter, 1 mL of a solution is sampled from the receptor after 3 hours, 6 hours, and 12 hours (1 mL of the PBS solution is added to the receptor after 3 hours and 6 hours). TPNa in the sampled solution is measured by HPLC (high performance liquid chromatography).

### <HPLC Analysis Conditions>

Column: CAPCELL PAK C18, Type MG 5 μm 4.6 I.D.×250 mm (available from Shiseido Co., Ltd.)

Mobile phase: (20 mM acetic acid+20 mM sodium acetate methanol)/acetonitrile=7/3

Flow rate: 1.0 mL/min

Temperature: 40° C.

Injection volume: 10 μl

The results of the above test are shown in Table 2.

TABLE 2

| | After 3 hours | After 6 hours | After 12 hours |
|---|---|---|---|
| Amount of skin | 0.08 | 0.10 | 0.14 |
| permeation (μg/cm²) | | | |

As shown in Table 2, the inventive external preparation may provide, after formation of the coating film, continuous transdermal administration of the active ingredient even though the surface of the coating film was washed 3 times. This is probably because the inventive base forms a hydrophobic coating film on the skin surface without the base being removed by repeated washing, which allows continuous transdermal administration of the active ingredient.

It is thus found that the inventive external preparation can, after formation of the coating film, transdermally administer the active ingredient over a long time while the inventive external preparation is not removed by bathing, face washing, or the like.

Since the formed coating film is not sticky, the coating film can undergo application of foundation thereon. The coating film can be thus covered with foundation in use. The formed coating film can be easily removed from the skin surface using cleansing oils or the like.

By the way, the inventive external preparation may optionally contain various additives other than the active ingredient, such as moisturizers, preservatives, antioxidants, and pH adjusters.

The active ingredient in the inventive external preparation is not limited to TPNa, and various active ingredients can be used according to the purpose of the inventive external preparation. Examples of active ingredients include corticosteroids, antiphlogistic analgesics, antihypertensive agents, anesthetics, sedative-hypnotics, tranquilizers, hypotensive agents, antibiotics, antibacterial substances, vitamins, antiepileptics, coronary vasodilators, antihistamines, antifungals, sublimate crystals, mentha oil, eucalyptus oil, lavender oil, boric acid solution, physiological saline solution, bitter water, linseed oil, lime water, liver oil, Rivanol solution, potassium permanganate solution, mentha water, creosote, mustard, anti-inflammatory agents, astringents, cooling agents, vitamin agents, hormonal agents, skin care agents such as antihistamines, sebum inhibitors, anti-acne drugs such as keratolytic drugs, animal or plant extracts, such as aloe extract, ginseng extract, and glycyrrhiza extract, and nutrients such as amino acids.

The present invention may be embodied in a variety of other forms without departing from the spirit or main features of the present invention. The foregoing examples are merely illustrative in every respect and should not to be construed as limiting. The scope of the present invention is defined by the claims and is not restricted by the description herein. All modifications and changes within the range of equivalents of the claims are within the scope of the present invention.

### INDUSTRIAL APPLICABILITY

The inventive base is suitably used as a base in an external preparation for skin (inventive external preparation) for transdermal administration of an active ingredient.

1. A base used in an external preparation for skin that administers an active ingredient transdermally, comprising at least an acrylic-based synthetic polymer and a volatile oil, wherein
the base is in a liquid or ointment form prior to application to a skin surface, and
the volatile oil evaporates after application of the base to form a hydrophobic coating film containing the volatile oil in the range of 0 to 60 wt % with respect to 100 parts by weight of the acrylic-based synthetic polymer.

2. The base according to claim 1, wherein
the base has hydrophobicity with a weight change rate (X) of 5.0% or less in accordance with the following test:
<Test>
where
the weight (D) of the base is measured after immersing a cotton nonwoven fabric (10 cm×10 cm, weight 60 g/m²) in the base and drying the fabric in a 60° C. thermostatic bath for 24 hours; and
the cotton nonwoven fabric after immersion and drying is next placed in water, stirred for 1 hour, and then taken out of water, and after removal of water on the surface of the fabric, the weight (W) of the fabric is measured and the weight change rate (X) is calculated in accordance with the following equation:
Equation: X=(W−D)×100/D.

3. The base according to claim 1, wherein
the acrylic-based synthetic polymer is at least one selected from alkyl acrylate copolymer, alkyl acrylate copolymer sodium, alkyl acrylate copolymer ammonium, (alkyl acrylate/diacetone acrylamide) copolymer, and (alkyl acrylate/dimethicone) copolymer.

4. The base according to claim 1, wherein
the volatile oil is at least one selected from short chain paraffins and volatile silicone oils.

5. An external preparation for skin, comprising:
the base according to claim 1; and
an active ingredient, wherein
the base is in a liquid or ointment form prior to application to a skin surface, and
the volatile oil evaporates after application of the base to form a hydrophobic coating film.

6. The external preparation for skin according to claim 5, for use in combination with application of foundation on a hydrophobic coating film formed by application of the external preparation to a skin surface.
